(19) United States
(12) Patent Application Publication
(10) Pub. No.: US 2006/0142669 A1
(43) Pub. Date: Jun. 29, 2006

(54) Urine sampling container

(75) Inventors: Ichiro Morimoto, Hyogo (JP); Akira Tsuji, Osaka (JP)

Correspondence Address: NIXON PEABODY, LLP, 401 9TH STREET, NW, SUITE 900, WASHINGTON, DC, 20004-2128, US

(73) Assignee: ATLETA INCORPORATION, Izumisano-shi, Osaka (JP)

(21) Appl. No.: 10/544,687

(22) PCT Filed: Feb. 12, 2003
(86) PCT No.: PCT/JP03/01425

§ 371 Date: Aug. 5, 2005

(51) Int. Cl. A61B 5/00

(52) U.S. Cl. 600/573

(57) ABSTRACT

A urine collection vessel includes: a narrow cylindrical container tube (2) for containing urine provided with an end closed and the other end opened; a narrow cylindrical urine collection tube (3) for collecting urine provided with an end closed, the other end which is opened and freely connected or disconnected to the opened end of the container tube (2) and a urine collection inlet (33) provided at the side of the urine collection tube; and a narrow cylindrical cover tube for closing the urine collection inlet (33) provided with an end closed and the other end opened and has a slightly larger diameter than the diameter of the urine collection tube (3) such that the urine collection tube (3) is freely inserted into and removed from the cover tube. Part of the urine collection tube (3) ranging from the urine collection inlet (33) to the closed end is formed as a measuring part (31) which contains and measures a predetermined amount of urine. Also included is a cap which closes the opened end of the container tube (2). The urine collection vessel further includes a protrusion (38) formed on the outer circumference surface of the urine collection tube (3) and a recess into which the projection (38) is fitted when the urine collection tube (3) is inserted into the cover tube. The urine collection tube (3) and the cover tube are simultaneously detached from the container tube (2).

### TECHNICAL FIELD

The present invention relates to a urine collection vessel used for urinalysis carried out in hospitals and examination centers.

### BACKGROUND ART

Various urinalyses have generally been carried out in hospitals and examination centers.

As an example of urine collection vessels used for the urinalyses, Publication of International Patent Application WO95/09362 discloses a urine collection vessel comprising a cylindrical vessel body having a urine collection inlet, a cap which is detachably attached to an opened end of the vessel body and a cover tube which is formed such that the vessel body is freely inserted therein and removed therefrom.

Further, as disclosed by Publication of International Patent Application WO95/09362, there is also another urine collection vessel comprising a cylindrical vessel body, a cap which is detachably attached to an opened end of the vessel body and provided with a urine collection inlet and a cover tube which is formed such that the vessel body is freely inserted therein and removed therefrom.

In collecting urine with these urine collection vessels, a test subject attaches the cap to the opened end of the vessel body and holds the cap such that the vessel body points downward. Then, urine is poured onto the urine collection inlet, thereby collecting the urine in the vessel body through the urine collection inlet. After the urine collection, the vessel body is inserted into the cover tube with the closed end of the vessel body kept pointing downward, thereby closing the urine collection inlet with the cover tube.

Problem to be Solved

The urine collection vessel described above is held in a vessel carrier and transferred to an examination room or the like with the urine collection inlet closed with the cover tube. However, the conventional urine collection vessel has a problem in that they complicate urinalysis to be executed in the examination room.

More specifically, according to an example of the urinalysis using the urine collection vessel, the cap is detached from the urine collection vessel with the vessel body kept inserted in the cover tube and then a reagent or the like is poured into the vessel body from the opened end of the vessel body. According to another example, the cover tube is detached from the urine collection vessel with the cap kept attached to the vessel body and a reagent or the like is poured into the vessel body from the urine collection inlet of the vessel body.

However, the conventional urine collection vessels are not equipped with a measuring means. Therefore, urine may be collected to fill the vessel up to the urine collection inlet of the cap and an extra amount of urine is difficult to dispose of. That is, in some cases, the amount of urine collected exceeds the capacity of the vessel body which will be solely placed in an examination apparatus. Therefore, it takes trouble in placing the vessel body in the examination apparatus.

In particular, if urine is collected in an excess amount, the urine in the vessel body may drip out of the cover tube through the opened end of the cover tube when the vessel body is inserted into the cover tube after the urine collection. This may possibly soil the hands of the test subject.

In view of the above, the present invention has been achieved. An object of the present invention is to allow collection of urine necessary for examination with reliability.

### DISCLOSURE OF THE INVENTION

According to a first invention, is provided a urine collection vessel comprising: a narrow cylindrical container tube for containing urine provided with an end closed and the other end opened; a narrow cylindrical urine collection tube for collecting urine provided with an end closed, the other end which is opened and freely connected or disconnected to the opened end of the container tube and a urine collection inlet provided at the side of the urine collection tube; and a narrow cylindrical cover tube for closing the urine collection inlet provided with an end closed and the other end opened and has a slightly larger diameter than the diameter of the urine collection tube so that the urine collection tube is freely inserted into and removed from the cover tube, wherein part of the urine collection tube ranging from the urine collection inlet to the closed end is formed as a measuring part which contains and measures a predetermined amount of urine.

According to a second invention, in relation to the first invention, the urine collection vessel further comprises a cap which closes the opened end of the container tube when the container tube and the urine collection tube are separated.

According to a third invention, in relation to the first or second invention, the urine collection vessel further comprises a connecting means for detachably connecting the urine collection tube and the cover tube.

According to a fourth invention, in relation to the third invention the connecting means includes a protrusion which is formed on the outer circumference surface of the urine collection tube and a recess which is formed on the inner circumference surface of the cover tube such that the protrusion is fitted in the recess when the urine collection tube is inserted into the cover tube.

According to a fifth invention, in relation to the first invention, the container tube is provided with a scale for indicating the amount of urine collected.

According to a sixth invention, the container tube, urine collection tube and cover tube of the first invention are sterilized.

According to a seventh invention, in relation to the first invention, a strip-shaped protrusion is formed on part of the outer circumference surface of the urine collection tube near the urine collection inlet.

### EFFECTS

In the present invention, first, the opened end of the container tube is connected to the urine collection tube to form an assembly ready for urine collection.

After the tubes are assembled, a test subject holds the container tube with the urine collection tube pointing downward. The test subject then pours urine onto the urine collection inlet such that the urine flows into the urine collection tube through the urine collection inlet and accumulates in the measuring part. Thus, urine collection is completed. During the urine collection, once a predetermined amount of urine (e.g., 10 ml) is infused into the measuring part, an additional amount of urine leaks out of the urine collection inlet of the urine collection tube. Therefore, an amount of urine required for urinalysis is surely collected without collecting extra urine.

When the urine collection is completed, the urine collection vessel is transferred to an examination room or the like. The transfer of the vessel may be carried out in two manners.

According to a first manner, the urine collection tube which is kept pointing downward is inserted into the cover tube. As a result, the urine collection tube which is a soiled part is covered. When the urine collection tube is inserted into the cover tube, the urine collection inlet is closed with the cover tube and the protrusion of the urine collection tube and the recess of the cover tube are engaged.

Then, the urine collection vessel is turned upside down, placed in a vessel carrier or the like with the container tube pointing downward and transferred to the examination room. At this time, since the urine collection vessel is turned upside down, the urine contained in the measuring part flows into the container tube and accumulates therein.

Then, the container tube is separated from the urine collection tube and the cover tube. At this time, the cover tube is pulled in a direction away from the container tube, whereby the cover tube and the urine collection tube which is kept connected to the cover tube by the connecting means are detached from the container tube at one time. More specifically, the urine collection tube and the cover tube are separated at the same time from the container tube with the protrusion and the recess engaged with each other. Then, only the container tube containing the urine is placed in an examination apparatus.

In a second manner, the container tube is transferred with the cap attached thereto. In this case, after the urine collection is completed, the urine collection tube is inserted into the cover tube with the urine collection tube pointing downward. By inserting the urine collection tube into the cover tube, the urine collection inlet is closed with the cover tube and the protrusion and the recess are engaged.

Then, the urine collection tube is turned upside down such that the urine contained in the measuring part flows into the container tube and accumulates therein. In this state, the cover tube is pulled in a direction away from the container tube to separate the cover tube and the urine collection tube which is kept connected to the cover tube from the container tube at one time. Then, the container tube containing the urine is sealed by attaching the cap to the opened end of the container tube, held in an upright position in a vessel carrier and then transferred to the examination room. In the examination room, the cap is removed from the container tube and the container tube is placed in an examination apparatus.

### EFFECT OF THE INVENTION

Thus, according to the present invention, the urine collection tube is provided with the measuring part. Therefore, an amount of urine required for the examination is surely collected. This eliminates the need of disposing of extra urine in the examination room. Therefore, the examination is carried out hygienically and quickly.

Since the urine collection tube is covered with the cover tube, a test subject is allowed to collect urine by merely pouring the urine onto the urine collection inlet. As a result, the urine collection is achieved in an exceedingly hygienic manner without spattering the urine on the hand.

Since the container tube does not have any openings, there is no need of transferring the urine into a centrifugation tube to subject the urine to centrifugation for examination. Therefore, the container tube is directly used in an examination apparatus, thereby speeding the examination.

If the cover tube and the urine collection tube are connected, the cover tube and the urine collection tube are simultaneously separated from the container tube without touching the soiled part. Therefore, the urine collection is achieved in an exceedingly hygienic manner.

Further, if the container tube is provided with the scale, the amount of urine collected is recognized with accuracy. Therefore, an examination record or the like is easily prepared.

If the container tube, urine collection tube and cover tube are sterilized, the kind and number of bacteria are examined with accuracy, thereby improving reliability of urinalysis.

Further, if the strip-shaped protrusion is formed on part of the outer circumference surface of the urine collection tube near the urine collection inlet, the urine is prevented from leaking into space between the urine collection tube and the cover tube.

### BRIEF DESCRIPTION OF DRAWINGS

FIG. 1 is a front view of a urine collection vessel of Example 1 of the present invention with a cover tube detached.

FIG. 2 is a vertical cross section of the urine collection vessel of Example 1 of the present invention with the cover tube attached thereto.

FIG. 3 is a vertical cross section of a container tube of Example 1 of the present invention with a cap attached thereto.

FIG. 4 is a vertical cross section of the container tube of Example 1 of the present invention.

FIG. 5 is a side view of a urine collection tube of Example 1 of the present invention.

FIG. 6 is a vertical cross section of the urine collection tube of Example 1 of the present invention.

FIG. 7 is a cross section illustrating Example 1 of the present invention taken along the line A-A shown in FIG. 5.

FIG. 8 is a vertical cross section of the cover tube of Example 1 of the present invention.

FIG. 9 is a side view of the cap of Example 1 of the present invention.

FIG. 10 is a front view of a urine collection vessel of Example 2 of the present invention before urine collection.

FIG. 11 is a vertical cross section of the urine collection vessel of Example 2 of the present invention after the urine collection.

FIG. 12 is an oblique view illustrating part of a urine collection tube of Example 2 of the present invention.

FIG. 13 is a front view of a urine collection vessel of Example 3 of the present invention before urine collection.

FIG. 14 is a vertical cross section of the urine collection vessel of Example 3 of the present invention after the urine collection.

FIG. 15 is a plan view of a urine collection tube of Example 3 of the present invention.

### BEST MODE FOR CARRYING OUT THE INVENTION

### EMBODIMENT 1

Hereinafter, with reference to the drawings, a detailed explanation is given of Embodiment 1 of the present invention.

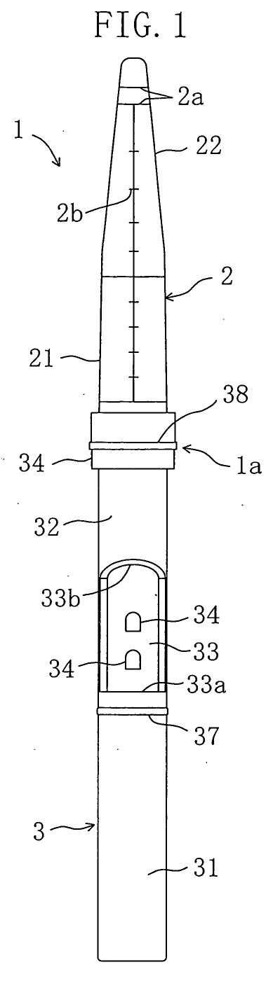

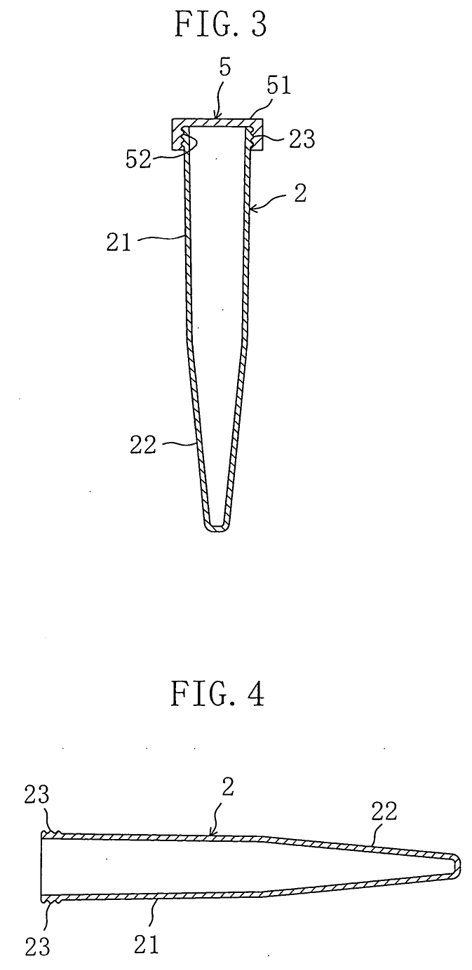

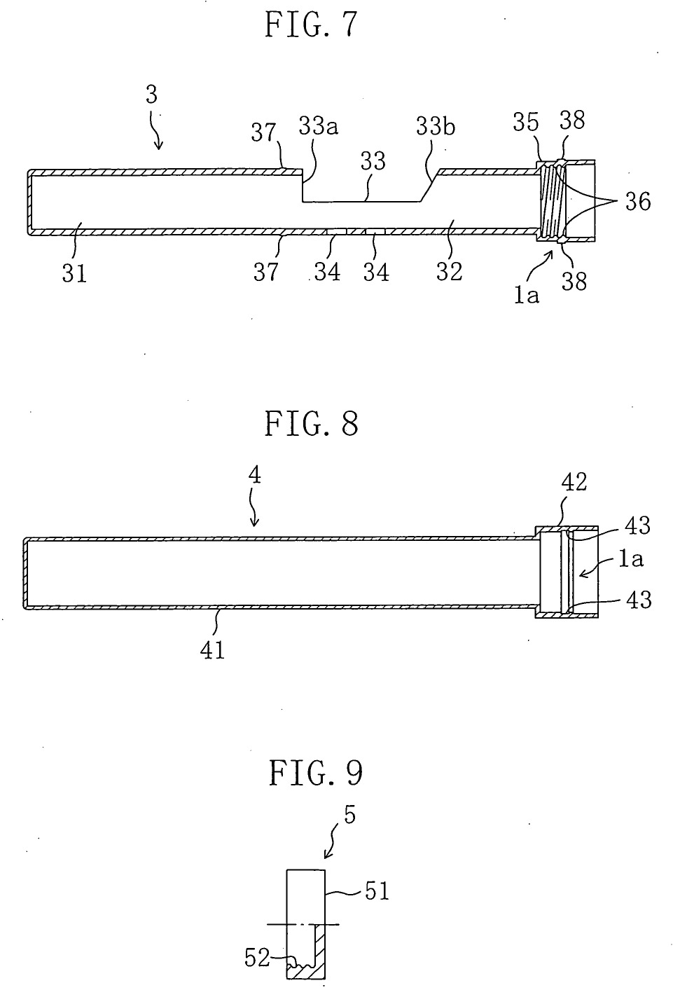

FIGS. 1 to 9 illustrate Embodiment 1 of the present invention. As shown in FIGS. 1 to 3, a urine collection vessel 1 is a vessel used for collecting urine for urinalysis carried out in hospitals and the like.

The urine collection vessel 1 includes a container tube 2 for containing urine collected, a urine collection tube 3 for collecting and measuring the urine and a cover tube 4 which is detachably attached to the urine collection tube 3.

As shown in FIG. 4, the container tube 2 is a narrow cylinder having an end closed and the other end opened. The container tube 2 is made of transparent material such as synthetic resin. The material for the container tube 2 is not limited to the transparent material and other materials such as colored translucent material may also be used.

The container tube 2 includes a main part 21 which is almost a left half thereof and a tapered part 22 which is almost a right half thereof as shown in FIG. 4. The container tube 2 is formed as a centrifugation tube having enough outer diameter and length to place the container tube 2 in a urinalysis apparatus.

The main part 21 of the container tube 2 is slightly tapered toward the tapered part 22. Threads 23 are formed on part of the outer circumference surface of the main part 22 near the opened end such that the urine collection tube 3 is fixed thereto. The tapered part 22 of the container tube 2 is continuous to the main part 21 and tapered toward the closed end thereof. The angle of the tapered part 22 of the container tube 2 is larger than the tapering angle of the main part 21.

At the tip portion of the tapered part 22 of the container tube 2, a first gauge line 2a and a second gauge line 2a are provided. When urine collected is subjected to centrifugation and the resulting supernatant is disposed of to obtain a urine sample for a urinary sediment examination (15 μl), the first gauge line 2a and the second gauge line 2a are used to indicate the amount of the remaining urine. The first and second gauge lines indicate 0.1 ml and 0.2 ml, respectively.

The container tube 2 is given with a scale 2b which indicates the amount of urine collected. The scale 2b is provided along the lengthwise direction of the container tube 2 so that the amount of urine collected is recognized.

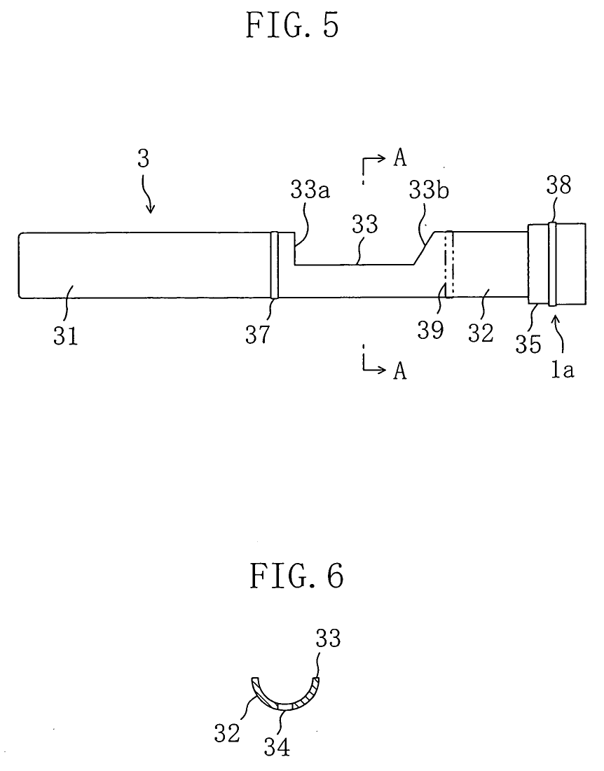

The urine collection tube 3 is a narrow cylinder having an end closed and the other end opened as shown in FIGS. 5 to 7. The urine collection tube 3 is made of translucent material such as synthetic resin. The material for the urine collection tube 3 is not limited to the transparent material and various materials such as non-tinted synthetic resin may also be used.

The urine collection tube 3 includes a measuring part which is almost a left half thereof and a urine collection part 32 which is almost a right half thereof as viewed in FIG. 5. The measuring part 31 has a closed end and configured to measure urine required for urinalysis. For example, if the measuring part 31 of the urine collection tube 3 is configured to have a volume of 10 ml, the measuring part 31 is formed into a cylinder of 13.8 mm in inner diameter and 69.00 mm in length. The volume of the measuring part 31 may be 15 ml or 20 ml. In short, the volume of the measuring part 31 may be 10 ml or more or less than 10 ml depending on the requirements of the examination and the like.

The urine collection part 32 of the urine collection tube 3 is a cylinder having an end continuous to the measuring part 31 and the other end opened. The urine collection part 32 is provided with a urine collection inlet 33 and auxiliary inlets 34. The urine collection inlet 33 is an opening which penetrates part of the wall of the urine collection tube 3 from the outer circumference surface to the inner circumference surface thereof and urine is poured thereon to collect the urine. The urine collection inlet 33 is formed by cutting away more than half of the circumference of the urine collection tube 3 and has a length of 30.00 mm in the axis direction.

The urine collection inlet 33 is formed to have an orthogonal plane 33a which is located at an end closer to the measuring part 31 and correspond with the direction of the radius of the urine collection tube 3 and an inclined plane 33b which is located at the other end and inclined toward the opened end of the urine collection tube 3. The measuring part 31 is defined as ranging from the orthogonal plane 33a of the urine collection inlet 33 to the closed end of the urine collection tube 3.

The auxiliary inlets 34 penetrate parts of the wall of the urine collection inlet 3 opposite to the urine collection inlet 33 from the outer circumference surface to the inner circumference surface thereof. The auxiliary inlets 34 are arranged in line along the axis direction. The number of the auxiliary inlet 34 may be one, or three or more. The auxiliary inlets 34 are formed to release air during the urine collection so that the urine collection is easily carried out and to prevent deformation during molding.

At the opened end of the urine collection part 32, a connecting part 35 to be connected to the container tube 2 is formed. The connecting part 35 has an outer diameter larger than that of the urine collection part 32 so that the opened end of the container tube 2 is freely inserted therein and removed therefrom.

The connecting part 35 is provided with threads 36 formed on the inner circumference surface thereof to be engaged with the threads 23 of the container tube 2. With the threads 23 of the container tube 2 and the threads 36 of the urine collection tube 6, the container tube 2 and the urine collection tube 6 are freely connected or disconnected. The threads 23 of the container tube 2 and the threads 36 of the urine collection tube 6 are so configured that the container tube 2 and the urine collection tube 6 are connected or disconnected relatively by a single rotation, ½ rotation or ¾ rotation.

The urine collection tube 3 includes a first protrusion 37 and a second protrusion 38 formed on the outer circumference surface thereof. The first protrusion 37 is in the form of a strip and formed along the entire circumference surface of the urine collection tube 3 to prevent backflow of the urine toward the end of the cover tube 4 and fix the cover tube 4. The second protrusion 38 is also in the form of a strip and formed along the entire circumference surface of the urine collection tube 3 to prevent the urine from leaking outside the cover tube 4 and fix the cover tube 4. The first protrusion 37 is formed on part of the outer circumference surface of the measuring part 31 near the urine collection inlet 33 and the second protrusion 38 is formed on part of the outer circumference surface of the connecting part 35. A component indicated by the chain double-dashed line shown in FIG. 5 is omitted in this embodiment.

The cover tube 4 is a narrow cylinder having an end closed and the other end opened as shown in FIG. 8. The cover tube 4 is made of colored material such as synthetic resin. The material for the urine collection tube 3 is not limited to the colored material and various materials may also be used.

The cover tube 4 has a slightly larger outer diameter than the diameter of the urine collection tube 3 such that the urine collection tube 3 is freely inserted therein and removed therefrom.

The cover tube 4 includes a cover main body 41 having enough length to close the urine collection inlet 33 and insert the urine collection tube 3 therein completely and a large-diameter part 42 which is formed at the opened end portion of the cover main body 41. On the inner circumference surface of the large diameter part 42, a recess 43 is formed such that the second protrusion 38 of the urine collection tube 3 is fitted therein.

The inner diameter of the cover main body 41 is determined such that the cover main body 41 contacts the first protrusion 37 of the urine collection tube 3 which slides thereinto, thereby preventing backflow of the urine flown out of the urine collection inlet 33 of the urine collection tube 3 toward the closed end of the cover tube 4. The recess 43 of the cover tube 4 is in the form of a strip and formed along the entire inner circumference of the cover tube 4 such that the second protrusion 38 of the container tube 2 is fitted therein when the urine collection tube 3 is inserted into the cover tube 4.

The recess 43 of the cover tube 4 and the second protrusion 38 of the urine collection tube 3 constitute a connecting means 1a. Therefore, to detach the container tube 2 from the urine collection tube 3 and the cover tube 4, the cover tube 4 is pulled in a direction away from the container tube 2, thereby detaching the cover tube 4 and the urine collection tube 3 which is kept connected to the cover tube 4 at one time from the container tube 2.

Further, the recess 43 of the cover tube 4 and the second protrusion 38 of the urine collection tube 3 constitute a sealing mechanism that prevents the urine from leaking out of the cover tube 4.

The urine collection vessel 1 described above includes a cap 5 for the container tube 2. The cap 5 includes a main body 51 which is shaped into an inversed U when viewed in section as shown in FIG. 9. The cap 5 further includes threads 52 which are formed on the inner circumference surface of a cylindrical part of the cap 5 and configured to be engaged with the threads 23 of the container tube 2. The cap 51 is so formed that the opened end of the container tube 2 is inserted therein and the threads 23 and 52 are engaged with each other to completely seal the container tube 2.

-Usage Directions for the Urine Collection Vessel 1-

Now, an explanation is given of usage directions for the urine collection vessel 11 described above.

First, for urine collection, the opened end of the container tube 2 is inserted into the connecting part 35 of the urine collection tube 3 and the threads 36 of the urine collection tube 3 and the threads 23 of the container tube 2 are engaged to connect the urine collection tube 3 and the container tube 2, thereby forming an assembly ready for urine collection.

A test subject holds the container tube 2 of the assembly thus formed with the urine collection tube 3 pointing downward. The test subject then pours urine onto the urine collection inlet 33 such that the urine flows into the urine collection tube 3 through the urine collection inlet 33 and accumulates in the measuring part 31. Thus, urine collection is completed. During the urine collection, once a predetermined amount of urine (e.g., 10 ml) is infused into the measuring part 31, an additional amount of urine leaks out of the urine collection tube 3 through the urine collection inlet 33. Therefore, an amount of urine required for the examination is surely collected without collecting extra urine.

After the urine collection is finished, the urine collection vessel 1 is transferred to an examination room or the like. The transfer of the vessel may be carried out in two manners.

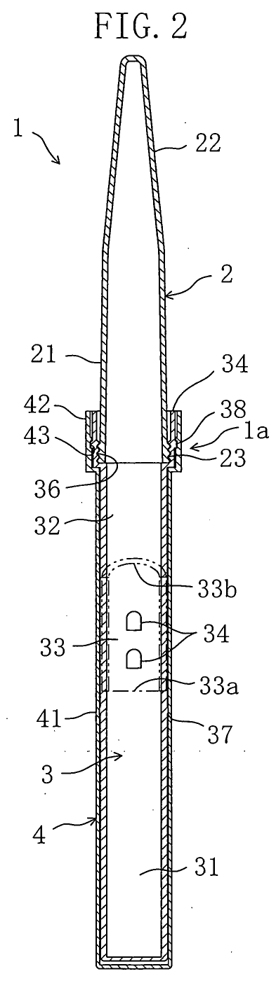

According to a first manner, as shown in FIG. 2, the urine collection tube 3 which is kept pointing downward is inserted into the cover tube 4. As a result, the urine collection tube 3 which is a soiled part is covered. When the urine collection tube 3 is inserted into the cover tube 4, the urine collection inlet 33 is closed with the cover tube 4 and the second protrusion 38 and the recess 43 of the cover tube 4 are engaged. The engagement of the second protrusion 38 and the recess 43 prevents the collected urine from leaking. Further, since the cover main body 41 and the first protrusion 37 of the urine collection tube 3 are in contact with each other, the urine flown out of the urine collection inlet 33 of the urine collection tube 3 will not flow toward the closed end of the cover tube 4. That is, the urine will not remain in the cover tube 4, thereby preventing shortage of the urine to be examined.

Then, the urine collection vessel 1 is turned upside down and held in a vessel carrier or the like with the container tube 2 pointing downward and transferred to the examination room. At this time, since the urine collection tube 3 is turned upside down, the urine contained in the measuring part 31 flows into the container tube 2 and accumulates therein.

Then, in the examination room, since the second protrusion 38 of the urine collection tube 3 and the recess 43 of the cover tube 4 are engaged, the cover tube 4 is rotated with respect to the container tube 2 to disengage the threads 23 and 36. With the threads 23 and 36 disengaged, the container tube 2 is detached from the urine collection tube 3 and the cover tube 4.

To detach the container tube 2 from the urine collection tube 3 and the cover tube 4, the cover tube 4 is pulled in a direction away from the container tube 2, thereby detaching the cover tube 4 and the urine collection tube 3 which is kept connected to the cover tube 4 by the connecting means la at one time from the container tube 2. Then, only the container tube 2 containing the urine is placed in an examination apparatus.

According to a second manner, the container tube 2 is transferred with the cap 5 attached thereto. Also in this case, after the urine collection is completed, the urine collection tube 3 which is kept pointing downward is inserted into the cover tube 4. By inserting the urine collection tube 3 into the cover tube 4, the urine collection inlet 33 is closed with the cover tube 4 and the second protrusion 38 and the recess 43 are engaged.

Then, the urine collection tube 3 is turned upside down so that the urine contained in the measuring part 31 flows into the container tube 2 and accumulates therein. In this state, the threads 36 of the urine collection tube 3 and the threads 23 of the container tube 2 are disengaged and the cover tube 4 is pulled in a direction away from the container tube 2 to separate the cover tube 4 and the urine collection tube 3 from the container tube 2 at one time. As a result, the container tube 2 containing the urine is isolated and the opened end of the container tube 2 is screwed into the cap 5 to seal the container tube 2. The sealed container tube 2 is then held in an upright position in a vessel carrier and transferred to the examination room. In this examination room, the cap 5 is detached from the container tube 2 and the container tube 2 is placed in the examination apparatus.

### EFFECT OF EMBODIMENT 1

As described above, according to this embodiment, the urine collection tube 3 is provided with the measuring part 31. Therefore, an amount of urine required for the examination is surely collected. This eliminates the need of disposing of extra urine in the examination room. Therefore, the examination is carried out hygienically and quickly.

Since the cover tube 4 and the urine collection tube 3 are connected, the urine collection tube 3 and the cover tube 4 are detached simultaneously from the container tube 2 without touching the soiled part. Therefore, urine collection is carried out in an exceedingly hygienic manner.

Since the urine collection tube 3 is covered with the cover tube 4, a test subject can carry out urine collection by merely pouring urine onto the urine collection inlet 33. As a result, the urine collection is achieved without spattering the urine on the hand, thereby allowing urine collection in an exceedingly hygienic manner.

Since the container tube 2 is not provided with any openings, there is no need to transfer the urine from the container tube into a spitz tube to subject the urine to centrifugation for examination. Since the container tube 2 is directly used in an examination apparatus, the examination is carried out quickly.

Further, since the container tube 2 includes the scale 2b, the amount of urine collected is recognized with reliability. Therefore, an examination record or the like is easily prepared.

### EMBODIMENT 2

Now, with reference to the drawings, a detailed explanation is given of Embodiment 2 of the present invention.

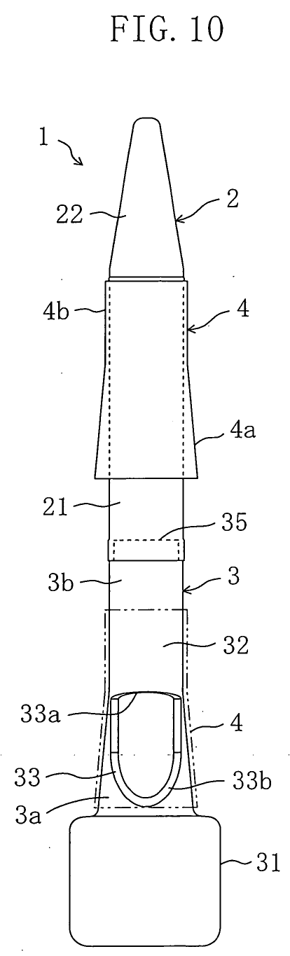

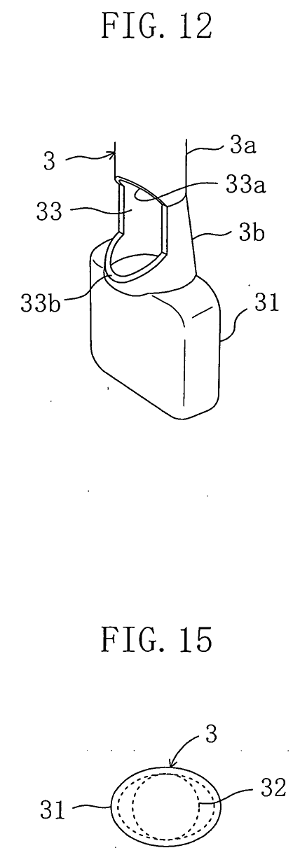

In this embodiment, the cover tube 4 of Embodiment 1 which completely covers the urine collection tube 3 is modified such that the cover tube 4 covers the urine collection inlet 33 only as shown in FIGS. 10 to 12.

More specifically, the measuring part 31 of the urine collection tube 3 is shaped into a rectangular tank. Then, a urine collection part 32 is continuously formed on the top surface of the measuring part 31. The urine collection part 32 includes a tapered part 3a and a perfect round part 3b. The tapered part 3a is shaped to increase in diameter in a downward direction in FIG. 10. The large-diameter end thereof is continuous to the measuring part 31 and the small-diameter end thereof is continuous to the perfect round part 3b. The tapered part 3a is provided with a urine collection inlet 33. The urine collection inlet 33 is formed to have an inclined plane 33b at the lower end thereof and an orthogonal plane 33a at the upper end thereof.

The perfect round part 3b is provided with a connection part 35 at the top end thereof as seen in FIG. 10 such that the container tube 2 is connected thereto.

The cover tube 4 includes a tapered part 4a and a perfect round part 4b to correspond with the urine collection part 32 of the urine collection tube 3. The tapered part 4a of the cover tube 4 is shaped to increase in diameter in a downward direction in FIG. 10 and the small-diameter end thereof is continuous to the perfect round part 4b. The perfect round part 4b of the cover tube 4 is fitted onto the container tube 2 and slides along the container tube and the perfect round part 3b of the urine collection tube 3.

When the cover tube 4 slides along the container tube 2 to the urine collection tube 3, the cover tube 4 is fitted with the tapered part 3a of the urine collection tube 3, thereby closing the urine collection inlet 33.

The cap 5 includes a small-diameter insert part 53 and a large-diameter flange 54. The insert part 53 is formed such that it is inserted in the opened end of the container tube 2 to seal the container tube 2.

Thus, for urine collection, as shown in FIG. 10, the opened end of the container tube 2 is inserted into the connection part 35 of the urine collection tube 3 to connect the urine collection tube 3 and the container tube 2, thereby forming an assembly ready for urine collection.

In this assembly, the cover tube 4 is positioned outside the container tube 2 and the urine collection inlet 33 is opened. A test subject holds the container tube 2 with the urine collection tube 3 pointing downward. The test subject then pours urine onto the urine collection inlet 33 such that the urine flows into the urine collection tube 3 through the urine collection inlet 33 and accumulates in the measuring part 31. Thus, urine collection is completed. During the urine collection, once a predetermined amount of urine (10 ml) is infused into the measuring part 31, an additional amount of urine leaks out of the urine collection tube 3 through the urine collection inlet 33. Therefore, an amount of urine required for urinalysis is surely collected without collecting extra urine.

After the urine collection is completed, the cover tube 4 which is not soiled is slid down such that the tapered part 4a of the cover tube 4 is fitted with the tapered part 3a of the urine collection tube 3, thereby closing the urine collection inlet 33 with the cover. Then, the urine collection vessel 1 is turned upside down while the vessel 1 is held at the cover tube 4. As a result, the urine contained in the measuring part 31 flows into the container tube 2 and accumulates therein.

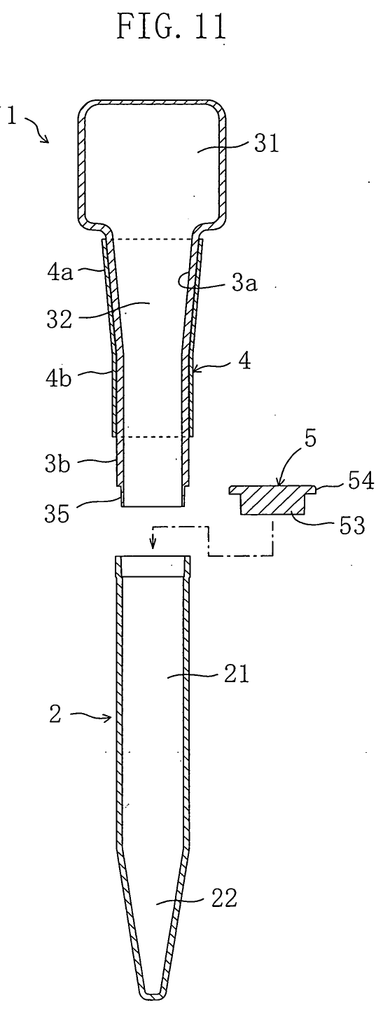

Then, in this state, the urine collection vessel 1 may be held in a vessel carrier and transferred to an examination room or the like. Or alternatively, as shown in FIG. 11, the urine collection tube 3 and the cover tube 4 may be removed from the container tube 2 and the opened end of the container tube 2 is closed with the cap 5. Then, the container tube 2 is held in the vessel carrier and transferred to the examination room. Other features, actions and effects than described above are the same as those of Embodiment 1.

### EMBODIMENT 3

Now, with reference to the drawings, a detailed explanation is given of Embodiment 3 of the present invention.

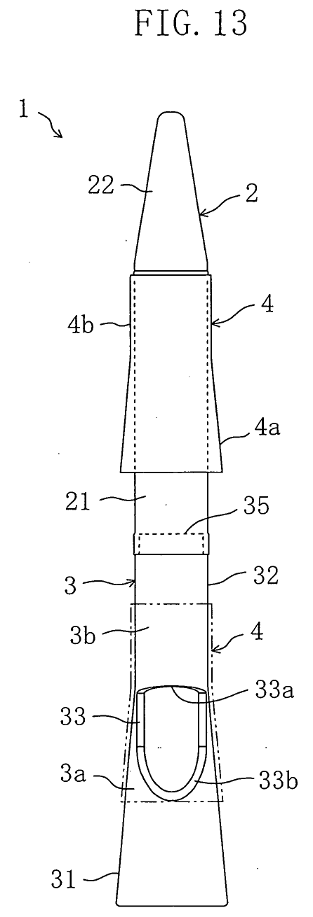

In this embodiment, the rectangular tank-shaped measuring part 31 of Embodiment 2 is modified into an oval cylinder as shown in FIGS. 13 to 15.

More specifically, the measuring part 31 of the urine collection tube 3 is continuous to a tapered part 3a of a urine collection part 32 and formed to increase in diameter in a downward direction in FIG. 13 and the large-diameter end thereof is closed. The small-diameter end at the top of the measuring part 31 is continuous to the large-diameter end of the tapered part 3a of the urine collection part 32.

Therefore, for urine collection, the opened end of the container tube 2 is inserted into a connecting part 35 of the urine collection tube 3 to connect the urine collection tube 3 and the container tube 2, thereby forming an assembly ready for urine collection.

In this assembly, the cover tube 4 is positioned outside the container tube 2 and the urine collection inlet 33 is opened. A test subject holds the container tube 2 with the urine collection tube 3 pointing downward. The test subject then pours urine onto the urine collection inlet 33 such that the urine flows into the urine collection tube 3 through the urine collection inlet 33 and accumulates therein. Thus, urine collection is completed. During the urine collection, once a predetermined amount of urine (10 ml) is infused into the measuring part 31, an additional amount of urine leaks out of the urine collection tube 3 from the urine collection inlet 33. Therefore, an amount of urine required for urinalysis is surely collected without collecting extra urine.

After the urine collection is completed, the cover tube 4 which is not soiled is slid down such that the tapered part 4a of the cover tube 4 is fitted with the tapered part 3a of the urine collection tube 3, thereby closing the urine collection inlet 33 with the cover. Then, the urine collection vessel 1 is turned upside down while the vessel is held at the cover tube 4. As a result, the urine contained in the measuring part 31 flows into the container tube 2 and accumulates therein.

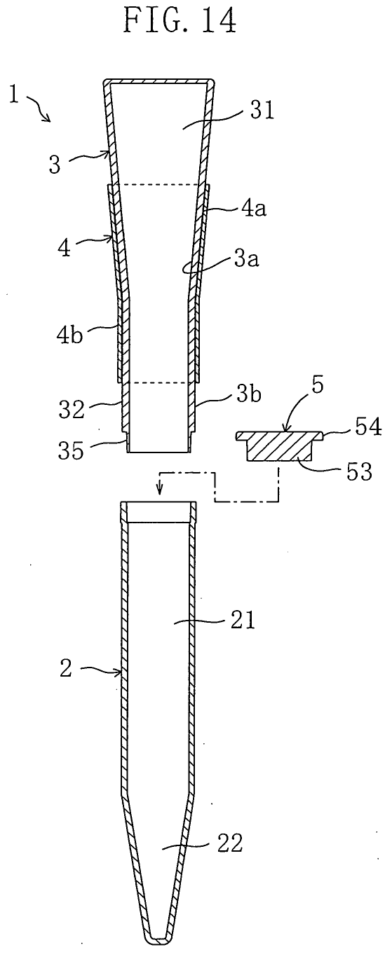

Then, in this state, the urine collection vessel 1 may be held in a vessel carrier and transferred to an examination room or the like. Or alternatively, as shown in FIG. 14, the urine collection tube 3 and the cover tube 4 may be detached from the container tube 2 and the opened end of the container tube 2 is closed with the cap 5. Then, the container tube 2 is held in the vessel carrier and transferred to the examination room. Other features, actions and effects than described above are the same as those of Embodiment 1.

### OTHER EMBODIMENTS

In the above embodiments, the container tube 2 and the urine collection tube 6 are provided with the threads 23 and 36, respectively. However, the threads 23 and 36 of the container tube 2 and the urine collection tube 6 may be a combination of a recess and a projection. In short, the container tube 2 and the urine collection tube 6 are formed such that they are connected or disconnected without leaking the urine out.

The container tube, urine collection tube and cover tube may be subjected to sterilization. The sterilization may be carried out using γ-ray, X-ray, β-ray, α-ray, electron beam or neutron beam.

If the container tube, urine collection tube and cover tube are sterilized, reliability of urinalysis improves because the kind and the number of bacteria are examined with accuracy.

As indicated by a chain double-dashed line in FIG. 5, the urine collection tube 3 may be provided with protrusions 37 and 39 formed on parts of the outer circumference surface thereof at both sides of the urine collection inlet 33. That is, on the outer circumference surface of the urine collection tube 3, a first protrusion 37 is formed on part of the measuring part 31 near the urine collection inlet 33 and a third protrusion 39 is formed at the side of the urine collection inlet 33 near the connecting part 35. The third protrusion 39 near the connection part 35 prevents the urine from leaking into space between the urine collection tube 3 and the cover tube 4 when the urine collection tube 3 inserted in the cover tube 4 is turned upside down.

### INDUSTRIAL APPLICABILITY

As described above, the urine collection vessel of the present invention is extremely useful as a urine collection vessel used for urinalysis in hospitals and the like.

1. A urine collection vessel comprising:
a narrow cylindrical container tube for containing urine provided with an end closed and the other end opened;
a narrow cylindrical urine collection tube for collecting urine provided with an end closed, the other end which is opened and freely connected or disconnected to the opened end of the container tube and a urine collection inlet provided at the side of the urine collection tube; and
a narrow cylindrical cover tube for closing the urine collection inlet provided with an end closed and the other end opened and has a slightly larger diameter than the diameter of the urine collection tube so that the urine collection tube is freely inserted into and removed from the cover tube, wherein
part of the urine collection tube ranging from the urine collection inlet to the closed end is formed as a measuring part which contains and measures a predetermined amount of urine.

2. A urine collection vessel according to claim 1 further comprising a cap which closes the opened end of the container tube when the container tube and the urine collection tube are separated.

3. A urine collection vessel according to claim 1 or 2 further comprising a connecting means for detachably connecting the urine collection tube and the cover tube.

4. A urine collection vessel according to claim 3, wherein the connecting means includes a protrusion which is formed on the outer circumference surface of the urine collection tube and a recess which is formed on the inner circumference surface of the cover tube such that the protrusion is fitted in the recess when the urine collection tube is inserted into the cover tube.

5. A urine collection vessel according to claim 1, wherein
the container tube is provided with a scale for indicating the amount of urine collected.

6. A urine collection vessel according to claim 1, wherein
the container tube, urine collection tube and cover tube are sterilized.

7. A urine collection vessel according to claim 1, wherein
a strip-shaped protrusion is formed on part of the outer circumference surface of the urine collection tube near the urine collection inlet.
